(19) United States
(12) Patent Application Publication
(10) Pub. No.: US 2014/0188090 A1
(43) Pub. Date: Jul. 3, 2014

(54) WOUND CARE ARTICLE

(71) Applicant: Birgit Riesinger, Munster (DE)

(72) Inventor: Birgit Riesinger, Munster (DE)

(21) Appl. No.: 14/070,154

(22) Filed: Nov. 1, 2013

Related U.S. Application Data
(63) Continuation of application No. PCT/EP2012/057980, filed on May 2, 2012.

(30) Foreign Application Priority Data
May 2, 2011 (DE) 10 2011 050 047.2
Jul. 3, 2012 (DE) 10 2012 101 912.6

(51) Int. Cl. A61F 13/58; A61F 13/02; A61M 27/00

(52) U.S. Cl. CPC A61F 13/58; A61M 27/00; A61F 13/0216
USPC 604/543; 604/385.03; 604/319; 604/365

(57) ABSTRACT

The invention concerns a wound care article, comprising at least one sheet-like layer, characterized in that the sheet-like layer has at least two wings and a central region, the wings being arranged around the central region and the wings being joined to each other via the central region.

### CROSS-REFERENCE TO RELATED APPLICATIONS

The present application claims priority from and is a continuation from PCT Application No. PCT/EP2012/057980, filed May 2, 2012, which claims priority from German Patent Application Serial No. DE 10 2011 050 047.2, filed May 2, 2011 and German Patent Application Serial No. DE 10 2012 101 912.6, filed Mar. 7, 2012 all of which are herein incorporated by reference in their entireties.

### BACKGROUND

The invention generally relates to a wound care article.

Wound care articles are known from the prior art, such as DE10059439 belonging to the applicant of the present application, with a body that absorbs the wound exudates. The latter has a sheet-like layer of absorption material, consisting of an absorbing nonwoven with superabsorber particles distributed therein.

These wound care articles have proven themselves to be exceptional in practical use, since they are able to take up exudates even from great depth and thus substantially improve the wound healing especially for chronic and/or edema-related wounds. However, these wound care articles are only suitable for relatively flat wounds.

Deep wounds, on the other hand, demand a wound filler material. For this, the wound must therefore be filled at first with a suitable filler and then the aforesaid wound care article is put in place. In many cases, this requires a difficult manipulation, which may impair the uptake capability of the wound care article and brings with it the problem of sepsis. Moreover, such a dressing can easily come apart.

Wounds in bodily regions with convex topology on the other hand require a flexible wound care article that can be put in place on the convex topology without causing wrinkles or exerting stresses or pressure.

The wound care articles mentioned above are only for wounds in the region of a relatively flat physical topology, thus for example for placement on legs or the chest. In regions with more complex physical topology (such as the heel or the sacrum), such wound care articles are not suitable for a successful wound treatment.

Wound care articles are known from DE 102009038387 A1 with a convex insert. These wound care articles have, besides a sheet-like layer that contains an absorbent material, at least one active element which gives the wound care article a convex or pyramidal form—in side view looking at its sheet-like orientation—and which is therefore suitable for the deeper wounds.

A wound care article is also known from DE102009043023 A1 that also has a sheet-like layer with absorbing material and an active element, the active element giving the wound care article a plastic deformability and shapability in certain areas, so that the wound care article under the action of a force is irreversibly deformed and thereby retains the corresponding shape.

Both in DE 102009038387 A1 and in DE102009043023 A1 the wound care articles contain additional active elements in order to generate the required shape of the wound care article.

The problem of the present invention is to provide an alternative wound care article that is suitable both for the treatment of deep wounds and for the treatment of wounds in regions of more complex physical topology.

This problem is solved with the features of the presented main claim.

### SUMMARY OF THE INVENTION

Provided herein are systems, methods and compositions for a wound care article, comprising at least one sheet-like layer, characterized in that the sheet-like layer has at least two wings and a central region, the wings being arranged around the central region and the wings being joined to each other via the central region.

The methods, systems, and apparatuses are set forth in part in the description which follows, and in part will be obvious from the description, or can be learned by practice of the methods, apparatuses, and systems. The advantages of the methods, apparatuses, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### BRIEF DESCRIPTION OF THE DRAWINGS

In the accompanying figures, like elements are identified by like reference numerals among the several preferred embodiments of the present invention.

FIG. 1 a shows a wound care article 100 according to the invention with a sheet-like layer 105, two wings 102, a central region 103 and a sheath 101. The sheet-like layer 105 has a round shape. The wings 102 can be obtained by incisions from the edge of the sheet-like layer 105 as far as the central region 103 of the sheet-like layer 105. The sheath 101 has a larger area than the sheet-like layer 105.

FIG. 1b shows a wound care article 100 according to the invention with a sheet-like layer 105, six wings 102, a central region 103 and a sheath 101. The sheet-like layer 105 has a round shape. The wings 102 can be obtained by incisions from the edge of the sheet-like layer 105 as far as the central region 103 of the sheet-like layer 105. The sheath 101 has a larger area than the sheet-like layer 105.

FIG. 2a shows a wound care article 100 according to the invention with a sheet-like layer 105, six wings 102, a central region 103, a sheath 101 and cutout regions 106. The cutout regions form part of the expansion space already mentioned above. The sheet-like layer 105 has a round shape. The wings 102 alternate with cutout regions 106. Wings 102 and cutout regions 106 are arranged alternating about the central region 103 of the sheet-like layer 105. The sheath 101 has a larger area than the sheet-like layer 105.

FIG. 2b shows a wound care article 100 according to the invention with eight wings 102 and eight cutout regions 106, a sheet-like layer 105, a central region 103 and a sheath 101. The wings 102 alternate with cutout regions 106. Wings 102 and cutout regions 106 are arranged alternating about the central region 103 of the sheet-like layer 105.

FIG. 3a shows a wound care article 100 according to the invention with two wings 102 of kidney shape. The central region 103 of the sheet-like layer 105, the cutout regions 106 and the sheath 101 are also shown.

FIG. 3b shows a wound care article 100 according to the invention with six wings 102, being incised laterally 107. The six wings 102 alternate with six cutout regions 106 and are uniformly arranged about the central region 103 of the sheet-like layer 105.

FIG. 4a shows a wound care article 100 according to the invention with a central region 103 and radial incisions 108 in the central region, six wings 102, six cutout regions 106, a sheet-like layer 105 and sheath 101.

FIG. 4b shows a wound care article 100 according to the invention with a central region 103 and radial incisions 108 in the central region, six wings 102 with lateral incisions in the wing 107, cutout regions 106, a sheet-like layer 105 and sheath 101.

FIG. 5a shows a wound care article 100 according to the invention with two wings 102 of kidney shape, a central region 103 and an opening 109 in the central region 103.

FIG. 5b shows a wound care article 100 according to the invention with three wings 102, a central region 103 and an opening 109 in the central region 103, with sheath 101 and cutout regions 106.

FIGS. 6a and 6b show the lining of a cavity 110 with a wound care article 100 according to the invention with folded-up wings 102, which partly overlap, and wings that may be bent at the incisions of the wings 107.

FIGS. 7a and 7b show the lining of a cavity 110 with an alternative wound care article 100 according to the invention. The wound care article shown in FIG. 7a corresponds to the types shown in FIG. 4a. One can see in particular the incisions 108 in the central region 103. The wound care article shown in FIG. 7b corresponds to the type shown in FIG. 2a.

FIGS. 8, 9, 10 and 11 show the placement of a wound care article 100 according to the invention in a bodily region with convex topology, namely, the knee (FIG. 8), the heel (FIG. 9), the breast (FIG. 10) and the elbow (FIG. 11). In this configuration, the wound care article according to the invention can be provided with an adhesive coating on the side facing the wound (in the manner of a border dressing). Alternatively, it can be fixed in place in the traditional manner, for example, with adhesive tape or the like.

FIGS. 12-21 show other possible embodiments, partly with a slot worked into the sheath or a recess (reference number 111) to place the wound care article around a catheter, a drainage tube or a cannula (FIG. 13) or for coupling together two or more wound care articles (FIG. 14a, FIG. 14b) by an overlapping. In departure from FIGS. 13 and 14, the sheath of the wound care article especially for the last mentioned purpose can also have several slots or recesses, as shown in FIG. 19, for example. Said slot or said recess can also be covered by a tear-off section (reference number 112 in FIGS. 15a and b), which is secured by means of perforations to the material of the sheath, so that it is only exposed by tearing off the section 112 when in use.

FIGS. 17-19 show that the sheet-like layer can also have incisions 113 in its central region. These facilitate the folding of the wound care article, for example, when arranging it in a ditchlike cavity. A similar situation is shown in FIG. 16, where one further notes that the wings of the sheet-like layer can be of different size.

FIGS. 20 and 21 show the radial incisions 114 can also be meandering or corrugated, either with parallel edges or with converging or diverging edges. In this way, when placed in a cavity or a bodily region with convex topology, it is ensured that fluid draining in the region of the incisions sooner or later touches the edges of the wings and is securely taken up.

FIGS. 22 and 23 show kits containing at least one wound care article 100 according to one of the preceding claims and at least one glue device. These glue devices can be configured as adhesive strips in the manner of a sticking plaster, being provided in the same or a different packaging than the wound care article. The adhesive strips can be elastic in configuration, have a pull-off protective film and an adhesive layer, such as one of silicone, acrylate, or hydrocolloidal adhesive. By means of the adhesive strips, which can also be fastened to the wound care article and/or form an integral part thereof, the wound care article can be attached to the skin of the patient. For this, the adhesive strips can be arranged in situ central (FIG. 22) or tangential or peripheral (FIG. 23) to the wound care article.

FIGS. 24 and 25 show wound care articles (100) according to the invention with radially symmetrical peripheral shape in the form of a square with rounded corners. This results in a kind of cloverleaf shape. In FIG. 24 the wound care article is shown without and in FIG. 25 with a sheath.

FIG. 25 shows the wound care article furthermore has incisions as far as the central region, although these can also be omitted.

FIG. 26 shows further possible applications of the wound care article of the invention.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

The subject of the invention is a wound care article comprising at least one sheet-like layer, characterized in that the sheet-like layer has at least two wings and a central region, the wings being joined to each other via the central region.

Said wound care article preferably has the shape of a propeller with at least two wings and a central region, the wings being arranged uniformly about the central region and the wings being joined to each other via the central region.

Said sheet-like layer has at least one material that is chosen from the group containing a mat, especially an airlaid mat, such as one having cellulose fibers, or a nonwoven of yarn or fibers of superabsorbent polymers with incorporated superabsorbing polymers, and/or a loose filling of superabsorbing polymers. Said airlaid mat can preferably have a sheet-like layer of absorption material, which consists of an absorbing nonwoven of the mentioned fibers with superabsorbing polymers distributed therein, for example.

The sheet-like layer—hereinafter alternatively termed an absorption body as well—can correspond to the absorbing insert, which is contained in a wound dressing of the applicant of the present invention, as is disclosed for example in WO03094813, WO2007051599 and WO0152780 and marketed under the brand name “sorbion sachet”. The disclosure content of the cited documents is incorporated in its entirety in the disclosure content of this document.

The sheet-like layer in another embodiment can likewise form a core, having (possibly flake-like) fibers or yarns of superabsorbing polymers as well as superabsorbing polymers in granulate form, wherein the granulates are glued or welded to the fibers or yarns on several levels, and the granulates are distributed over more than 50% of the entire structural height of at least one section of the core, with mixed regions of granulate and fibers being present. The weight percentage of the superabsorbing polymers can preferably lie in the range of 10 to 25 wt. %. Similar constructions are known from traditional incontinence materials and are known as sanitary napkins on account of their cushioning properties. A sheath can be arranged about said core, which is overlapping in regions, and which is covered by a glue seam, for example, or forms a part of the latter.

The sheet-like layer in another embodiment can likewise contain at least one flat layer having fibers or yarns of superabsorbing polymers, to which superabsorbing polymers in granulate form are glued. This produces, in one preferred embodiment, a construction of the body having at least three layers, with two cover layers surrounding a layer having superabsorbing polymers. There are no mixtures of fibers and superabsorbing polymers in the sheet, but only the two materials fixed and adjacent to each other.

The term “wound care article” shall mean hereinafter in particular a wound dressing, preferably a flat wound dressing or a wound care cloth. Said wound dressing contains a sheet-like layer and can be either absorbing or nonabsorbing, or only slightly absorbing. In particular, the term “wound care article” can also mean an ensemble of different products that are placed in a given arrangement on the wound being treated. This ensemble can form a physical unit, the different products being combined in a common sheath or adhesively joined to each other—possibly without a sheath. The ensemble, however, can also be present in the form of a kit, wherein the different products are placed in the given arrangement on the wound being treated by means of a compress.

The term “form of a propeller” means that the sheet-like layer has a shape in which at least two wings are arranged about a central region, like the blades of a helicopter propeller about a central shaft. The wings are oriented preferably radially, i.e., in a star pattern, about the central region. The wings can be situated in the same plane as the central region.

According to the invention, the wound care article is designed preferably for treatment of deep wounds, wounds in cavities or wounds in bodily regions with convex or irregular topography.

The term “cavity” means an existing or newly formed hollow in a tissue or organ or a deep wound.

The term “bodily region with convex topology” is self-explanatory. However, it means not only bodily regions with permanently convex topology, such as the heel, the chin, the back of the head, the shoulder, the hip, the breast, individual fingers or the fist, but also bodily regions with temporarily convex topology, such as knee or elbow joints. When applied to such bodily regions with convex topology, the wound care article of the invention is placed in a concave form.

The term “bodily region with irregular topology”, on the other hand, shall designate bodily regions such as those having regions with cavities and regions with convex topology.

The term “deep wound” includes all wounds that are not merely superficial.

When positioning the wound care article in a cavity, the wings can stick out from the plane of the central region, for example, they can be folded up or folded down as compared to the plane of the central region in order to achieve an optimal lining of the cavity. In this way, the wound care article of the invention can be adapted especially effectively to the topology of the wound, the organ, or the tissue. Accordingly, the wound care article is placed in the immediate vicinity of the wound, the organ or the tissue and cavities are avoided or at least minimized between wound, organ or tissue and wound care article.

Yet the wound care article of the invention is also especially advantageous when placing the wound care article on a bodily region with convex topology, since the configuration of the invention prevents parts of the wound care article from overlapping, which would reduce the wearing comfort and possibly cause friction and other consequences.

But this configuration can also be in wounds in bodily regions with irregular or even planar topology. For the former case, the wound care article has a greater flexibility and thus snugness, while for the latter case it has benefits in regard to the uptake capacity (on this, see the discussion in regard to the expansion spaces provided by the incisions) and the rate of uptake of fluids is increased, since a larger entry and contact area is provided between the sheet-like layer and the fluid being taken up.

One particular embodiment of the wound care article is characterized in that the central region has a radially symmetrical peripheral shape, the wings being produced by radial incisions that extend from the margin of the sheet-like layer to the central region. Said incisions form part of the expansion space to be discussed below.

By “radially symmetrical peripheral shape” is meant hereinafter those shapes for which a rotation through a certain angle about an axis of rotation brings the object back again to overlap itself. The axis of rotation here runs through the centroid of the shape. One speaks of an n-fold radial or rotational symmetry when a rotation about 360°/n mirrors the object on itself. Thus, the term encompasses not only the circular shape, but also the shape of an equilateral triangle (3-fold), the shape of a square (4-fold), or the shapes of polygons (especially a pentagon (5-fold), hexagon (6-fold) and octagon (8-fold)). It is important, in particular, that in the case of triangles, squares and polygons the corners themselves can be rounded. Thus, for example, one gets a cloverleaf shape in the case of a square (see figures).

In another configuration the central region has the shape of a regular polygon, the wings being produced by radial incisions that extend from the margin of the sheet-like layer to the central region. The central region can have the shape of a regular polygon with 2 to 20 or more corners, such as the shape of a triangle, square, pentagon, hexagon, heptagon or octagon.

In practice, the claimed shape can be made by processing a sheet-like layer, the claimed shape being created by radial incisions and/or recesses. Before the processing, said sheet-like layer can preferably take on a rotationally symmetrical basic shape, such as the basic shape of a circle, a square, or a regular polygon (also known as a regular n-gon).

In this embodiment of the wound care article of the invention, the propeller blades have an especially large area. This facilitates the lining of large cavities, for example. A further benefit of propellers with large area is that the wings can partially overlap when lining the cavity. In this way, a complete lining of the cavity can be achieved. Furthermore, in this way a larger quantity of wound care article can be placed in the cavity, so that more exudate can be taken up, for example.

The term “exudate” means a wound fluid that is derived from blood plasma through the inflammation process of the wound edema. Just as the blood is responsible for the transport of nutrients and other messenger substances and thus for supplying various parts of the body, the exudate in an entirely similar manner serves to supply the wound bed and the healing processes taking place in it. To handle these many functions, it contains a broad spectrum of components which results in a specific gravity that is slightly higher than that of water. In this it differs from the transudate, which is derived from non-inflammatory processes and has a distinctly lower specific gravity with a low content of cells and protein. Besides furnishing nutrients for the fibroblasts and epithelial cells, the exudate coordinates the various processes of wound healing in time and in space by its high content of growth factors and cytokines. These are formed primarily by thrombocytes, keratinocytes, macrophages and fibroblasts. They influence the motility, migration and proliferation of the various cells involved in the wound healing. Thus, migration of cells into the wound base is promoted, as is the supplying of the newly formed granulation tissue by angiogenesis. The wound cleaning is also supported by the exudate. It contains various serine, cysteine and aspartate proteases, as well as matrix metalloproteases, which are strictly regulated in their activity and break down irreversibly damaged tissue, thereby preparing the wound bed for the subsequent phases of healing.

In particular, physiological exudate contains salts, glucose, cytokines and growth factors, plasma proteins, proteases (especially matrix metalloproteases), granulocytes and macrophages.

In another embodiment of the wound care article of the invention, the number of wings is chosen from the group consisting of 2 to 20 or more wings, especially 3 wings, 4 wings, 5 wings, 6 wings, 7 wings, 8 wings, 9 wings, 10 wings, 11 wings, 12, wings, 13, wings, 14, wings, 15 wings, 16 wings, 17 wings, 18 wings, 19 wings or 20 wings.

With a larger number of wings, a more complicated topology can be more easily modeled.

In another embodiment of the wound care article of the invention regions are cut out from the sheet-like layer between the individual wings, so that wings and cutout regions are alternately arranged uniformly about the central region of the propeller.

Due to the cutout regions between the wings, wound care articles according to the invention are provided that contain less material for the sheet-like layer.

This embodiment can be advantageous under certain circumstances, for example, it may be easier to manipulate the arranging of the wings in a cavity, since the wings will overlap comparatively less or not at all. Furthermore, if the wings are folded up for example, small holes are formed between the central plane and the wings, through which exudate can drain or be suctioned off, or through which medicine can be placed in the cavity. The cutout regions can have the same size as the wings, but they can also be larger or smaller than them. Moreover, the cutout regions provide an expansion space, which is available to the absorbent material for an increase in its volume when taking up fluids.

This embodiment is likewise advantageous when arranging the wound care article on a bodily region with convex topology, since it prevents parts of the wound care article from overlapping, which would reduce the wearing comfort and cause friction or other consequences.

In another embodiment of the wound care article of the invention the wings and, if cutout regions are present, the wings and/or the cutout regions have a conical shape. Thanks to the conical shape, the wings can be especially easily adapted to and line cavities with a conical shape, such as wounds that are large at the surface and small on the inside. A placement on bodily regions with convex topology is also advantageous.

In another embodiment of the wound care article of the invention the wings are kidney shaped. The propeller shape generated in this way is then especially advantageous, for example, when the wound care article has only a few propeller wings, such as two wings. The wound care article according to the invention can be arranged in the cavity so that the wings line the cavity as completely as possible in the upper region, while the sheet-like layer may even overlap in the region of the wings, and gaps remain in the lower region of the cavity through which exudate can drain or be suctioned off, for example.

In another embodiment of the wound care article of the invention at least one wing has one or more lateral incisions in the wing. In another embodiment of the wound care article of the invention all wings have one or more lateral incisions in the wings.

Lateral incisions in the wings facilitate the bending of the winds in particular sites, so that the wound care article of the invention can be easily adapted as optimally as possible to the topology of the cavity. The lateral incisions can all be located on the same side of the wings or on different sides of the wings. The lateral incisions can be located only in the upper part, in the middle part, or in the lower part of the wings, the lower part of the wings being the part of the wings that is closest to the central region. In one embodiment of the wound care article of the invention, each wing has one lateral incision roughly in the middle of the wing.

In another embodiment of the wound care article of the invention the central region has at least one radial incision in the central region. The wound care article according to the invention can also have several radial incisions in the central region. In another particular embodiment of the wound care article according to the invention the central region has at least one radial incision in all cutout regions between the wings. The wound care article according to the invention can also have several radial incisions in one or more cutout regions. The number of the radial incisions in the cutout regions can be different within a wound care article according to the invention, for example, one cutout region between two wings can have one incision, while another cutout region between two other wings of the same wound care article can have two incisions.

The incisions in the central region of the wound care article facilitate the folding or bending in the central region. Thanks to this, the wound care article can also be overlapped in the central region. If necessary, material of the sheet-like layer can also be spread apart in the central region thanks to the incisions in the central region.

The incisions in the central region and in the region of the wings increase the adapting possibilities and the form fitting of the wound care article of the invention for cavities. Thanks to the number of wings, the choice of the wing shape, the presence or not of cutout regions between the wings and the shape of the cutout regions, the presence or not of incisions in the wings and/or in the central region, as well as the arrangement and number of the incisions, the wound care article according to the invention can be optimized for different shapes of cavities. In general, this not only improves the handling of the wound care article as compared to known wound care articles, but also makes the wound care better, since the wound care article according to the invention can be optimally adapted to the topology of the cavity and the required treatment.

In another embodiment of the wound care article of the invention the central region has an opening, preferably a central opening.

Through an opening in the central region of the wound care article according to the invention one can place, for example, instruments and treatment materials on the wound, the organ or the tissue. Through an opening in the central region it is possible to introduce hoses, for example. An opening in the central region can be used to put a drainage device in place. Through the opening in the central region a surgical conduit can be led. Through an opening in the central region medication can be administered.

In an especially preferred embodiment of the invention, the wound care article according to the invention comprises absorbent material. For example, the sheet-like layer consists of absorbent material.

In another embodiment of the wound care article of the invention the absorbent material has a nonwoven, preferably an airlaid fabric, and/or the absorbent material has superabsorbent polymers.

The term “nonwoven” means a textile sheet of individual fibers which, unlike woven, knitted and crocheted fabrics, is not made from yarn. Nonwovens maintain their structural integrity as a rule by the clinging of the individual fibers to each other. They can be made by filling, for example.

The term “airlaid” denotes a nonwoven of cellulose and polyolefin fibers, in which superabsorbent polymers are embedded.

“Superabsorbent polymers” (SAP) are plastics which are able to take up liquids by a multiple of their natural weight—up to 1000 times. Chemically speaking, they are a copolymer of acrylic acid (propenic acid, C3H4O2) and sodium acrylate (the sodium salt of acrylic acid, NaC3H3O2), while the ratio of the two monomers to each other can vary. In addition, a so-called core cross linker (CXL) is added to the monomer solution, which joins together the resulting long-chain polymer molecules in places by chemical bridges (“cross linking” them). Thanks to these bridges, the polymer becomes water insoluble. When water or aqueous salt solutions get into the polymer particle, it swells up and stiffens this network on the molecular level, so that the water can no longer escape effortlessly.

Alternatively, superabsorbers can be chosen based on methyl acrylic acid, polyvinyl alcohol/maleic anhydride copolymers, polysaccharide/maleic anhydride copolymers, maleic acid derivates, acrylamidopropane sulfonic acid copolymers, starch/acrylonitrile graft polymers, gelatinized starch derivates, alkyl or hydroxyalkyl cellulose, carboxymethylcellulose, starch/acrylic acid graft polymers, vinylacetate/acrylate copolymers, acrylonitrile or acrylamide copolymers.

The superabsorber particles can be present in powder or granulate form in a particle size between 100 and around 1000 μm.

But the fiber form is likewise especially preferred for superabsorbent polymers, since this is a very soft product both in the dry and in the swollen state, which can be shaped and is not stiff, and which furthermore has a slight abrasiveness, unlike the superabsorbent polymers in granulate or powder form. This holds for both fibers as such and for fiber woven, knitted or unwoven fabrics and/or fiber wadding.

These properties make superabsorbent polymers in fiber form especially suitable for products according to the invention that are configured as patient beds and bedsore cushions. Good softness is especially important in this area of application.

Furthermore, as the applicant has observed, superabsorbent polymers in fiber form have a faster response to fluids than superabsorbent polymers in granulate or powder form. Thus, superabsorbent polymers in fiber form support the wick effect of the recesses according to the invention.

However, cellulose derivates can also be preferred for said superabsorbent polymers:

Sulfoalkylated cellulose and its derivates, preferably cellulose ethylsulfonates

Carboxyalkylated cellulose, preferably carboxymethylcellulose, carboxyethylcellulose and/or carboxypropylcellulose

Complex cellulose derivatives, such as sulphoethylcarboxymethylcellulose, carboxymethylhydroxyethylcellulose, hydroxy-propyl-methylcellulose, and

Amidated cellulose derivates, such as carboxymethylcellulose amide or carboxypropylcellulose amide.

But alginates can also act as said superabsorbent polymers. Said superabsorbent polymers can also be hydrogel nanoparticles having hydroxy-terminated methacrylate monomers, such as 2-hydroxyethylmethacrylate (HEMA) and/or 2-hydroxypropylmethacrylate (HPMA), which are marketed as Altrazeal, for example.

The superabsorbent polymers can be present in the wound care article of the invention in the form of a granulate, a powder, a loose quantity, a pressed piece, a foam, in the form of fibers, a fiber weaving, knitting, or nonwoven and/or a fiber wadding. For example, the absorbent material can consist of 40 wt. % or more of superabsorbent polymers.

The wound care article according to the invention comprises a sheet-like layer, which contains an absorbent material. Especially preferred here is an essentially sheet-like layer of absorbent material that consists of an absorbing nonwoven with superabsorbent polymers distributed therein.

The absorbent material is preferably configured such that it absorbs the wound exudate as efficiently as possible. The absorbent material for example has a weight per unit of area of at least 300 g/m², the mass per unit of area of the osmotically active substances uniformly distributed therein being at least 100 g/m².

The absorbent material preferably has a material that is chosen from the group containing a mat, especially one made of an airlaid of said yarns or fibers of superabsorbent polymers with superabsorbent polymers worked in, and/or a loose fill of superabsorbent polymers. Said airlaid mat can preferably have an essentially sheet-like layer of absorption material, consisting for example of an absorbing nonwoven of the mentioned fibers with superabsorbent polymers distributed therein.

The absorbent material can be an absorbent insert, which is contained in a wound dressing of the applicant of the present invention and which is disclosed for example in WO03094813, WO2007051599 and WO0152780 and marketed under the brand name “sorbion sachet”. The disclosure content of the cited documents is enclosed in its entirety in the disclosure content of this document.

The absorbent material in another embodiment can likewise form a core, which has—possibly flake-like—fibers or yarns of superabsorbent polymers as well as superabsorbent polymers in granulate form, the granulates being glued or welded to the fibers or yarns on several levels, and the granulates being distributed over more than 50% of the total structural height of at least one section of the core, mixed regions of granulate and fibers being present. The weight percentage of the superabsorbent polymers can preferably lie in the range of 10 to 25 wt. %. Similar constructions are known from traditional incontinence materials and are familiar as sanitary napkins dressings for their cushioning properties.

The absorbent material in another embodiment can likewise contain at least one flat layer having fibers or yarns of superabsorbent polymers, to which superabsorbent polymers in granulate form are glued. This produces in one preferred embodiment a composition of the body having at least three layers, where two cover layers surround a layer having superabsorbent polymers. There are no mixtures of fibers and superabsorbent polymers present in the sheet, but rather the two materials are only fixed in place and adjacent to each other. Several layers can be provided and in one preferred embodiment they can be physically compacted together by rolling, pressing, calendering or other methods. Furthermore, the body can have repeating patterns or textures, such as a check pattern, a punched pattern, or the like.

The nonwoven or the superabsorbent polymers as part of the wound care article of the invention can be placed quite close to the action site. By contrast, the wound fillers described thus far in the prior art are generally passive, i.e., they are not outfitted with active working agents. The proximity of wound and absorbent material created with the help of the wound care article according to the invention is especially advantageous, since superabsorbent polymers can bind proteins and bacteria, as is shown for example in DE102007054127 of the applicant of the present invention. The closer it is placed to the wound, therefore, the more advantageously does this attribute work on the wound healing.

One embodiment of the wound care article according to the invention furthermore has at least in some sections a sheath that is permeable to fluid at least in some sections.

Said sheath can have, for example, the rotationally symmetrical basic shape, such as the shape of a circle, a square, or a regular polygon. The sheath as well as the sheet-like layer can also mimic the mentioned incisions and/or cutouts of the sheet-like layer.

One or more sheaths can be arranged around the absorbent material and overlap in certain regions, and cover the one glue seam or a portion thereof, for example. A section of a hydrophobic and/or water-repellent or water-impermeable material can likewise be provided inside the sheath, acting as a protection against wetness or protection of the bed linen.

The at least partially fluid-permeable sheath surrounds the sheet-like layer, forms a barrier to solid excretions, and allows the passage of other eliminated substances to the sheet-like layer of absorption material arranged inside the sheath. The sheath is preferably closed off at least partly by a seam.

Especially preferably, the sheet-like layer has an area in top view on its flat side which is 3% to 75% smaller, in its nonwetted condition, than the area of the sheath laid flat and is freely movable or fixed in the sheath. In this way, an expansion space is provided. This ensures that the sheet-like layer can increase in its volume upon taking up fluid and is not restricted by the sheath. The sheath is preferably configured so as to have pores which are smaller on average than the nonwetted superabsorbent polymers.

Especially preferably, the sheath has in top view on its flat side a raised section above the seam and the absorbent material is free of hard, sharp edges and corners.

The pores or mesh of the sheath are preferably about 0.05 mm to about 1.0 mm, advantageously about 0.20 mm to about 0.50 mm in size. Furthermore, the pores or mesh can preferably be bounded by the thread or fiber sections which are somewhat curved in cross section through the sheath and have their curved legs pointing outward.

The sheath is preferably formed from woven or fleecelike plastic fibers, such as polypropylene or polyethylene fibers, but also cotton, silk or rayon. It consists preferably of a weaving or nonwoven having a weight per unit area of at least 20 g/m². Preferably, the sheath consists of hydrophobic material, or the sheath material has a hydrophobic finish. The hydrophobic properties of the sheath prevent sticking to the wound surface and help the wound exudate particles get more quickly into the inside of the sheath.

In an embodiment of the invention the sheath has an elastic material at least in some sections, such as fibers of Lycra or Elasthan. In this way, the absorbent material can increase in volume when taking up fluid and is not restricted by the sheath

The material of the sheath can be structurized so that the sheath has a rough inner surface and a smooth outer surface. Preferably, the rough inner surface of the sheath is formed by funnel-like perforations, which taper in the direction of the inner surface and emerge into a free opening edge (“overhang”). This rough inner surface prevents displacements of the contents of the sheath, so that a fixation with glue spots is not needed. Accordingly, the smooth outer surface of the sheath material can be formed by arched sections of material extending between the perforations. Such a sheath material can be termed “three dimensional”, as opposed to one that is flat on both sides, and is known from, say, DE102006017194 of the applicant of the present invention, the disclosure content of which is here referenced in its entirety.

Moreover, the sheath in top view can have a rotationally symmetrical basic shape, for example, a circle, a square, or a regular polygon. Likewise, the sheath just like the sheet-like layer can mimic the mentioned incisions and/or cutouts of the sheet-like layer.

Especially preferably, said three-dimensional sheath material is laminated onto the aforementioned polypropylene nonwoven. Such a configuration results in improved fluid uptake properties.

It can likewise be provided that the wound care article of the invention has on its side away from the wound a fluid-impermeable protection for the bed linen (“backsheet”) at least in some sections. Furthermore, the sheath can be furnished with a physiologically acceptable adhesive (such as a hydrocolloid adhesive) on the side facing the wound.

In one arrangement of the wound care article in cavities it can make sense to do without such a sheath or to configure the sheath so that it mirrors the aforementioned incisions and/or cutouts of the sheet-like layer. When the wound care article is arranged on bodily regions with convex topology, it can likewise make sense to do without such a sheath or to configure the sheath so that it mirrors the aforementioned incisions and/or cutouts of the sheet-like layer.

It can also be provided that the wound care article according to the invention has, for example on its side facing the wound, at least in some sections, a sheath material with hydroactive polymers. This can be, e.g., a layer of material containing modified cellulose.

The modified cellulose is preferably cellulose derivatives, especially sulfoalkylated cellulose and its derivates, especially cellulose ethylsulfonates, carboxyalkylated cellulose, especially carboxymethylcellulose, carboxyethylcellulose and/or carboxypropylcellulose, more complex cellulose derivatives such as sulphoethylcarboxymethylcellulose, carboxymethylhydroxyethylcellulose, hydroxy-propyl-methylcellulose, and amidated cellulose derivates such as carboxymethylcellulose amide or carboxypropylcellulose amide. Carboxymethylcellulose is available in particular in the form of sodium carboxymethylcellulose and is marketed under the name “Hydofaser”. In sanitary and wound care products, the fibers are led into a flat matrix. By uptake of fluid from the wound exudate, the fibers are gradually transformed into a gel cushion, which retains the fluid and does not release it. The fibers are of such a construction that the wound exudate is taken up only in the vertical direction. This means that, as long as capacity remains, the exudate will not flow beyond the edge of the wound. In this way, maceration of the wound edge can be effectively prevented.

Said hydroactive polymers can also be alginates. Alginates are obtained from brown algae and are woven into a fibrous fleece. Chemically speaking, they are polysaccharides, namely, calcium and/or sodium salts of alginic acids. Alginates can absorb up to 20 times their own weight in fluid, in which process the wound exudate is stored in the voids. The Ca2+ ions contained in the alginate lattice are exchanged for the Na+ ions from the exudate until the level of saturation of Na ions in the alginate is reached. This results in a swelling of the wound dressing and a transformation of the alginate fibers into a gel body due to swelling of the fibers.

Said hydroactive polymers can likewise be hydrogel nanoparticles having hydroxy-terminated methacrylate monomers, such as 2-hydroxyethylmethacrylate (HEMA) and/or 2-hydroxypropylmethacrylate (HPMA), which are marketed for example as Altrazeal.

This embodiment is especially preferable for a wound care article that is to be placed in a cavity, such as a fistula, since said hydroactive polymer can then form a gel in the wound region and be perceived by the patient as pleasant and pain-relieving. A sticking is also prevented.

In another embodiment, the absorption body has a percentage of ≧40 wt. % of superabsorbent polymers. Especially preferably, the weight percentage of the superabsorbent polymers is ≧45, 50, 55, 60, 65 or 70 wt. %. Wound care articles with such high weight percentages of superabsorbent polymers have an especially good uptake capability for fluids and can sometimes replace the vacuum-style wound care systems on the market.

In further embodiments of the invention, the wound care article of the invention has at least one active ingredient with nutritive and/or disinfectant or decontaminating and/or protease-inhibiting action and/or a complex of active ingredients and/or a hemostatic agent.

Hemostatics are chemically and/or physiologically working ingredients or ingredient complexes with at least one substance or a composition of substances having blood clotting properties.

The active ingredient and/or complex of ingredients with disinfectant action can be, for example, a composition of at least one vitamin or vitamin derivate, a metal ion, and a detergent. Likewise, it can be a BLIS (bacteriocin like inhibitory substance) or coated magnetic particles.

The active ingredient and/or complex of ingredients with nutritive action can be a composition containing at least the components of an enteral and/or parenteral dietetic. Likewise, it can be at least one active ingredient chosen from the group containing insulin, recombinant insulin, proinsulin, an insulin-like growth factor (IGF), an insulin mimetic and/or a diabetic-specific energy carrier not based on glucose or saccharose.

The active ingredient and/or complex of ingredients with protease inhibiting action can be at least one active ingredient chosen from the group containing protease inhibitors, chelating agents for divalent cations, collagens, coated magnetic particles, acids, buffers, nonpathogenic acid-producing microorganisms, probiotics and/or symbiotics.

Further relations and background information on the active ingredients and/or ingredient complexes with nutritive, disinfectant or decontaminating and/or protease-inhibiting action are specified in DE102007030931 of the applicant of the present application, whose content is here referenced in its entirety. DE102007030931 also specifies other active ingredients and/or ingredient complexes with nutritive, disinfectant or decontaminating and/or protease-inhibiting action, which are likewise deemed as being disclosed in this application.

For example, the wound care article of the invention can comprise at least one active ingredient and/or ingredient complex with nutritive, disinfectant or decontaminating and/or protease-inhibiting action.

Moreover, it is preferable for the flat layer to have a pattern of incisions and/or punchings. The incisions and/or punchings here are preferably configured and/or arranged so that the entry of fluid into the wound care article is facilitated.

Moreover, it is preferable for the wound care article of the invention to have, at least in some sections, at least one heavy metal that is present in elemental or ionic form, preferably chosen from the group containing copper, zinc and/or silver. Said elemental or ionic heavy metal can be present in colloidal form, in superficial form, in a form bound to or covering fibers or particles, or the like.

If is furthermore preferable to provide the wound care article of the invention with an adhesive coating on the side facing the wound (in the manner of a border dressing). For this, one can use, for example, a polyacrylate adhesive, a silicone adhesive, or a hydrocolloid adhesive.

Moreover, it is preferable that the wound care article according to the invention have at least one slot in its sheath. In this way, the wound care article can be placed around a catheter, a drainage tube, or a cannula, or used to couple two or more wound care article to each other.

The subject of the invention is also a wound care system having one or more wound care articles according to the invention. In one embodiment of the invention, the wound care system comprises one or more wound care articles according to the invention and a drainage device and/or a surgical conduit.

The invention also pertains to the use of a wound care article according to the invention for the treatment of deep wounds, of bodily regions with convex topology and/or of wounds situated in the region of an irregular bodily topology.

The wound care article according to the invention can easily be adapted to the topology and shape of the wound being treated. In the case of a deep wound, the wound care article according to the invention can be placed in a shape, by folding up the wings and arranging the wings in the cavity, so that the wound care article fills out the wound and comes in contact with the wound bed over broad areas. In this way, deep edema and accumulations of exudate are also reached, and the wound healing process is promoted.

The wound care article of the invention when used in cavities makes it possible to dispense with other wound fillers that are used in the prior art (see above). Additional active elements to create and stabilize the shape of the wound care article for cavities are also unnecessary.

However, additional active elements can be used to support the adapting of the shape of the wound care article of the invention to the cavity of the wound, the organ, or the tissue.

One embodiment of the invention relates to a wound care article in which the wound care article has at least one additional active element, such as one that is described in DE 102009038387 A1or in DE102009043023 A1. The wound care article of the invention can be placed in a convex (for cavities) or concave shape (for bodily regions with convex topology), which is then stabilized by an additional active element, for example.

Furthermore, all combinations of concave and convex shaping of the wound care article are of course conceivable to adapt the wound care article of the invention to every conceivable anatomical circumstance. It is preferable for said active element to have a reinforcement of plastically deformable or shapable material.

By “reinforcement” is meant in the following a strengthening layer which imparts a plastic deformability or shapability to the wound care article, but is not manifested on the surface of the wound care article and consequently also cannot make contact with the wound. For example, the plastically deformable or shapable material can be a wire or tape material, especially a plastic or paper tape, on which two wires are embedded at the side for reinforcement. The plastically deformable or shapable material can comprise at least one material chosen from the group containing metal, plastic, hydrocolloid material, foam, dental modeling compound and/or bioplastics.

The wound care article of the invention can furthermore be configured so as to be able to wrap around a surgically implanted conduit. For this, the wound care article can have at least one slot, for example, making it possible to place the dressing on the patient's body around a conduit (such as a drainage conduit or a catheter), the wound care article being coordinated with a second, likewise sheet-like wound care article that is situated at a distance from the first wound care article, the distance being spanned by a connection strip or web. Such a wound care article is known, for example, from DE202006005966 of the applicant of the present invention, whose content is added in its entirety to the disclosure content of the present specification.

The invention also concerns the use of a wound care article (100) according to one of the preceding claims in a partial vacuum wound care system.

Such wound care systems are known, e.g., from DE202004017052, WO2006048246 and DE202004018245 of the applicant of the present invention. Express reference is made to the disclosure content.

From DE202004017052 a device is known for wound treatment making use of a partial vacuum, that a gas-tight wound covering element, which in the state when placed on the body of the patient forms a space remaining between the wound and the wound covering element, and at least one connection site that is in contact with the space and by which the air located in the space can be evacuated, wherein the wound covering element has underneath it at least one sheet-like wound care article taking up the wound secretions, whose volume increases over the course of the absorption process, so that the absorbed wound secretions remain inside the wound care article and thus underneath the wound covering element until the removal of the wound care article from the patient's body, the wound care article being at least one layer of a textile section fortified with superabsorbents and surrounded by a fluid-permeable sheath, and the layer in top view has an area on its flat side that is 3% to 90% smaller than the sheath, so that the wound care article can approximate a circular shape in cross section close to its total filling capacity.

From WO2006048246 there is known a multicomponent dressing for treatment of wounds of the human or animal body making use of partial vacuum, having: a wound covering element for placement on skin and mucous membrane surfaces, at least one connection site that is in contact with the wound space and by which the substances situated in the wound space can be evacuated, having superabsorbing polymers, and the absorbed wound secretions remain bound to polymers in the wound space until removed from the wound space, the polymers by their binding capacity supporting mutual synergies with the subatmospheric pressures.

From DE202004018245 there is known a drainage device for wound treatment making use of partial vacuum, having a gas-tight wound covering element consisting of filmlike material, which in the condition placed on the patient's body is adhesively secured about the wound region on the skin surface and forms a sealed space remaining between the wound and the wound covering element, at least one drainage tube that can be placed in the space, by which the substances located in the space can be evacuated, and at least one wound care article arranged inside the space and absorbing the wound secretions, having at least one layer of a textile section fortified with superabsorbents, surrounded by a fluid-permeable sheath, wherein the absorbed wound secretions remain inside the wound care article and thus beneath the wound covering element until the wound care article is removed from the patient's body, and wherein the wound covering element has a treatment opening that can be closed gas-tight, through which the wound care article can be inserted into the space and removed from the space.

An advantage of the wound care article according to the invention is that it is not stiff, despite optimal adapting to the topology, and therefore it follows the movements of the patient. The wound care article of the invention therefore means a substantial increase in patient comfort and avoids problems such as skin irritation and wound chafing.

Further benefits and advantageous embodiments of the subjects of the invention shall be illustrated by the drawings and explained in the following description. It should be noted that the drawings are merely of a descriptive nature and are not meant to restrict the invention in any way.

There are shown:

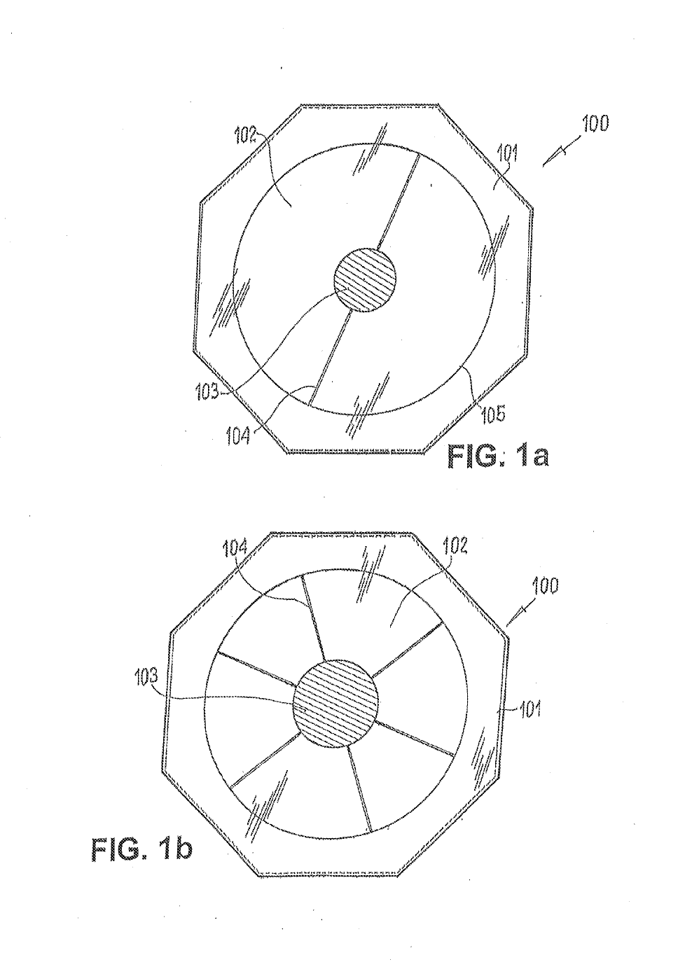

FIG. 1 a shows a wound care article 100 according to the invention with a sheet-like layer 105, two wings 102, a central region 103 and a sheath 101. The sheet-like layer 105 has a round shape. The wings 102 can be obtained by incisions from the edge of the sheet-like layer 105 as far as the central region 103 of the sheet-like layer 105. The sheath 101 has a larger area than the sheet-like layer 105.

FIG. 1b shows a wound care article 100 according to the invention with a sheet-like layer 105, six wings 102, a central region 103 and a sheath 101. The sheet-like layer 105 has a round shape. The wings 102 can be obtained by incisions from the edge of the sheet-like layer 105 as far as the central region 103 of the sheet-like layer 105. The sheath 101 has a larger area than the sheet-like layer 105.

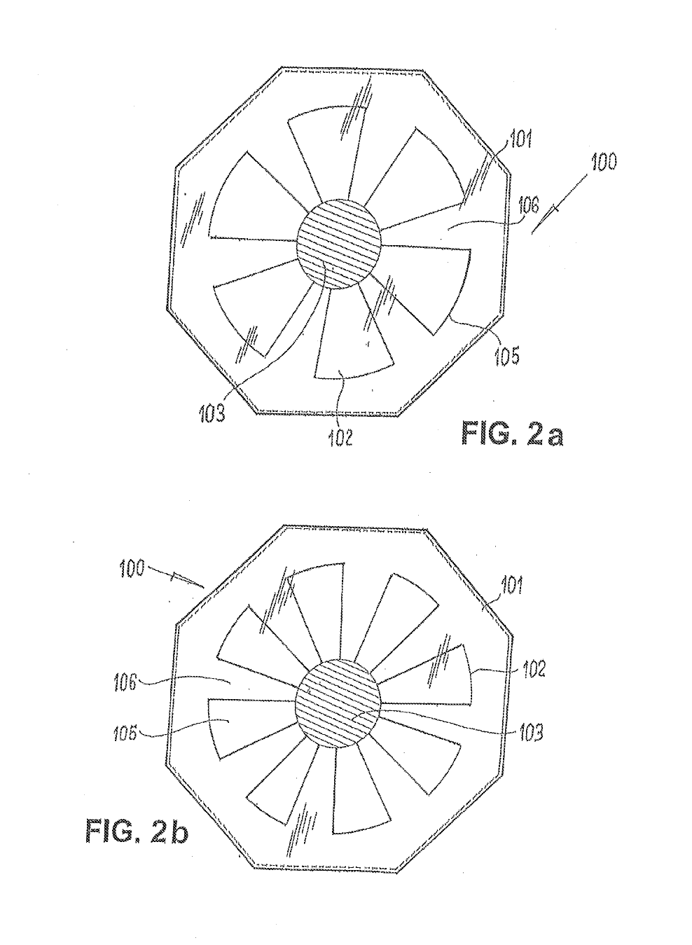

FIG. 2a shows a wound care article 100 according to the invention with a sheet-like layer 105, six wings 102, a central region 103, a sheath 101 and cutout regions 106. The cutout regions form part of the expansion space already mentioned above. The sheet-like layer 105 has a round shape. The wings 102 alternate with cutout regions 106. Wings 102 and cutout regions 106 are arranged alternating about the central region 103 of the sheet-like layer 105. The sheath 101 has a larger area than the sheet-like layer 105.

FIG. 2b shows a wound care article 100 according to the invention with eight wings 102 and eight cutout regions 106, a sheet-like layer 105, a central region 103 and a sheath 101. The wings 102 alternate with cutout regions 106. Wings 102 and cutout regions 106 are arranged alternating about the central region 103 of the sheet-like layer 105.

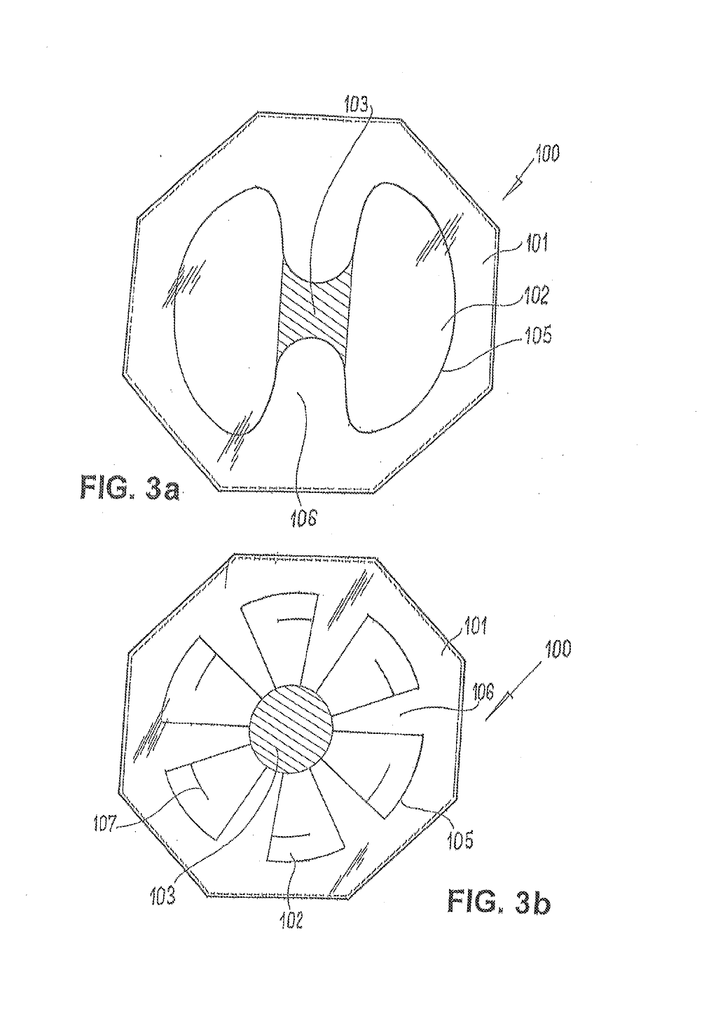

FIG. 3a shows a wound care article 100 according to the invention with two wings 102 of kidney shape. The central region 103 of the sheet-like layer 105, the cutout regions 106 and the sheath 101 are also shown.

FIG. 3b shows a wound care article 100 according to the invention with six wings 102, being incised laterally 107. The six wings 102 alternate with six cutout regions 106 and are uniformly arranged about the central region 103 of the sheet-like layer 105.

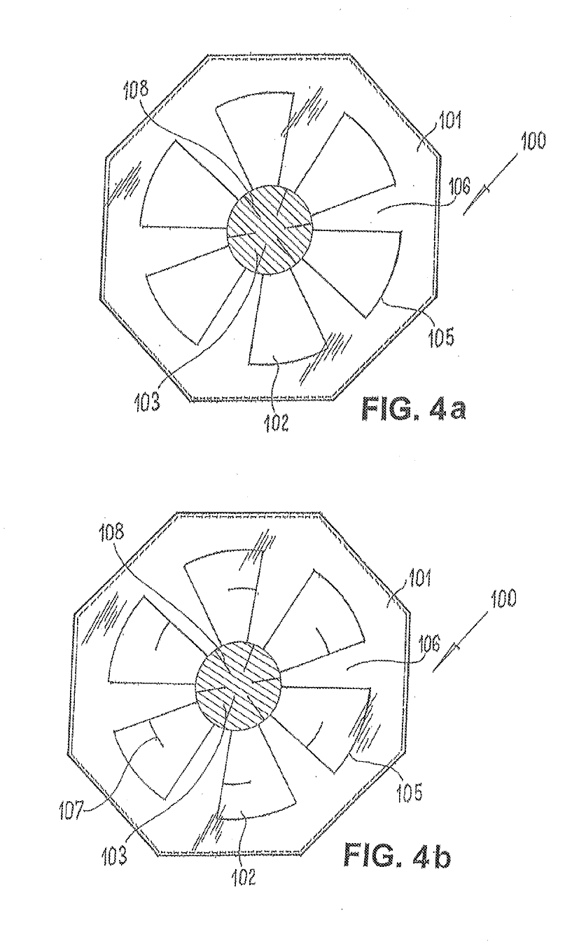

FIG. 4a shows a wound care article 100 according to the invention with a central region 103 and radial incisions 108 in the central region, six wings 102, six cutout regions 106, a sheet-like layer 105 and sheath 101.

FIG. 4b shows a wound care article 100 according to the invention with a central region 103 and radial incisions 108 in the central region, six wings 102 with lateral incisions in the wing 107, cutout regions 106, a sheet-like layer 105 and sheath 101.

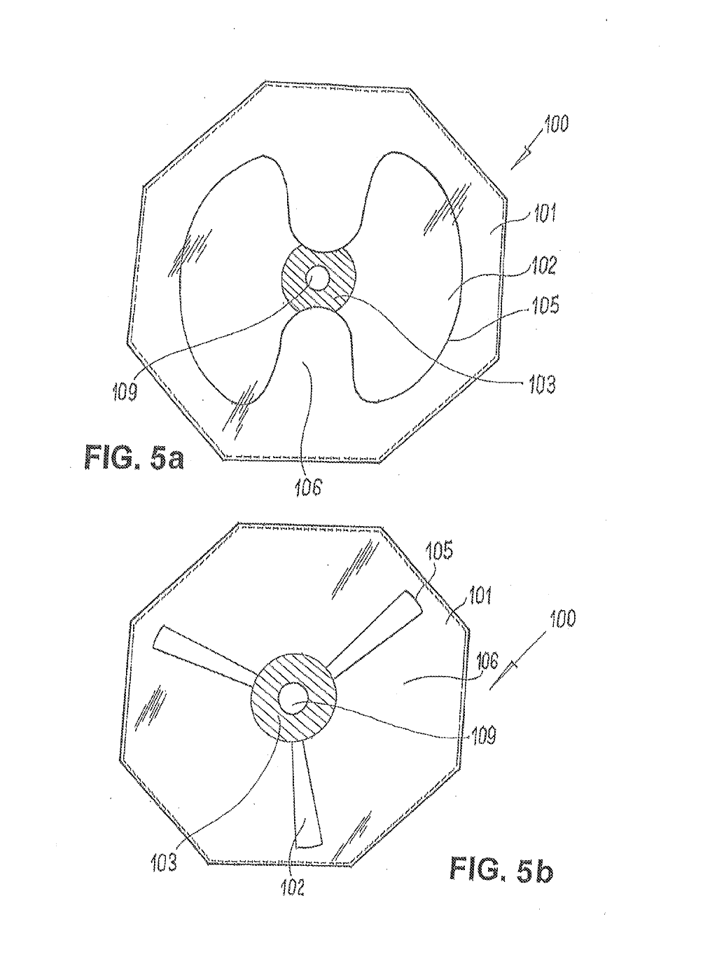

FIG. 5a shows a wound care article 100 according to the invention with two wings 102 of kidney shape, a central region 103 and an opening 109 in the central region 103.

FIG. 5b shows a wound care article 100 according to the invention with three wings 102, a central region 103 and an opening 109 in the central region 103, with sheath 101 and cutout regions 106.

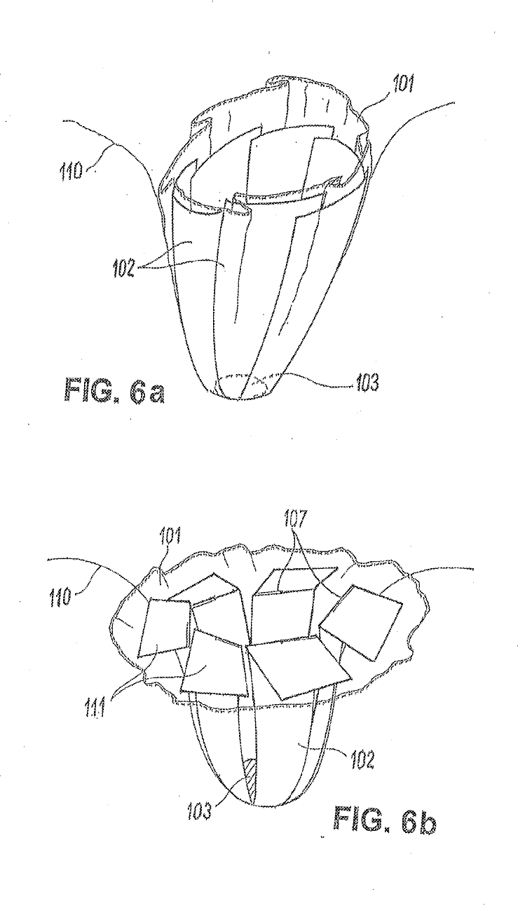

FIGS. 6a and 6b show the lining of a cavity 110 with a wound care article 100 according to the invention with folded-up wings 102, which partly overlap, and wings that may be bent at the incisions of the wings 107.

This ensures a firm purchase of the wound care article of the invention on the mentioned part of the body, and thus the wound care article is able to make contact with the wound being treated over its entire area.

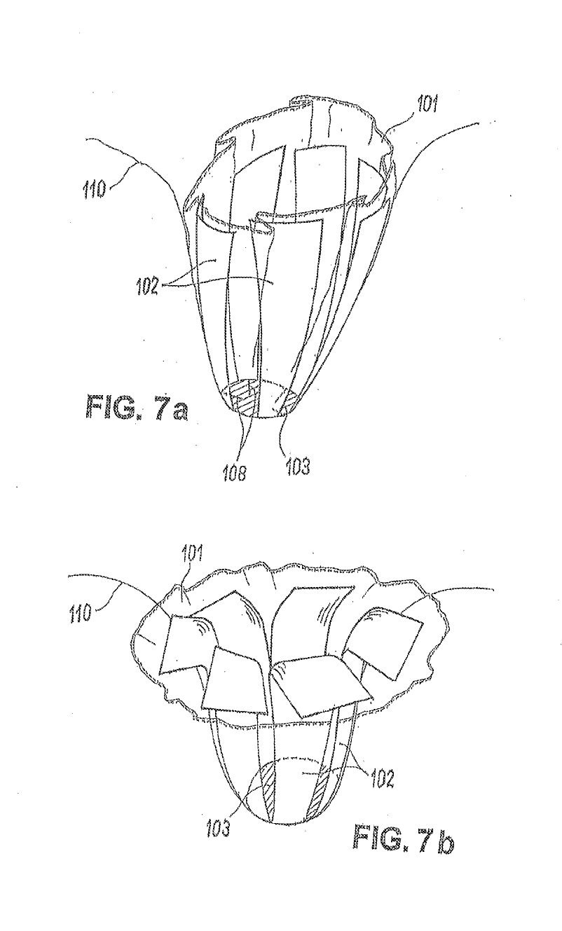

FIGS. 7a and 7b show the lining of a cavity 110 with an alternative wound care article 100 according to the invention. The wound care article shown in FIG. 7a corresponds to the types shown in FIG. 4a. One can see in particular the incisions 108 in the central region 103. The wound care article shown in FIG. 7b corresponds to the type shown in FIG. 2a.

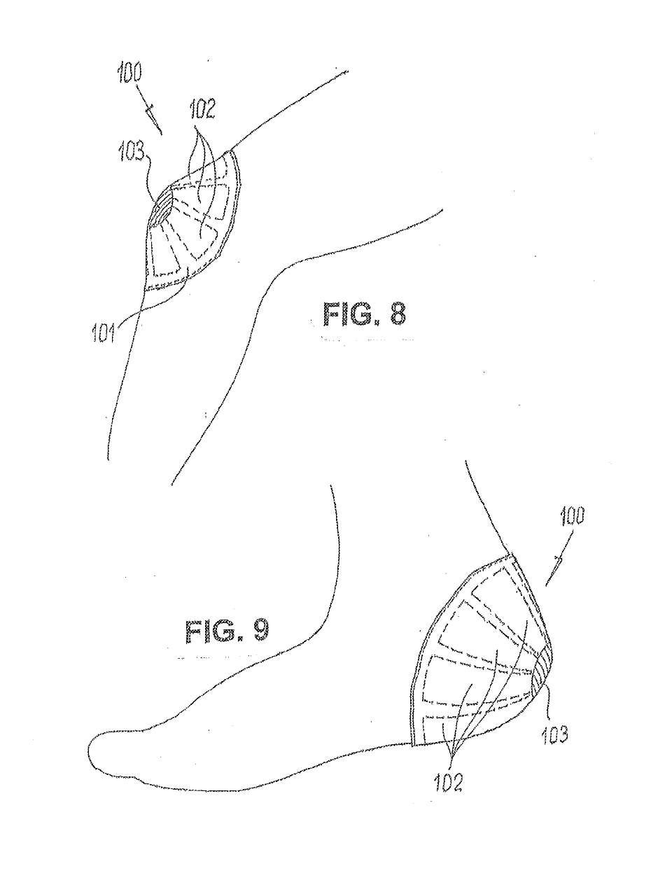

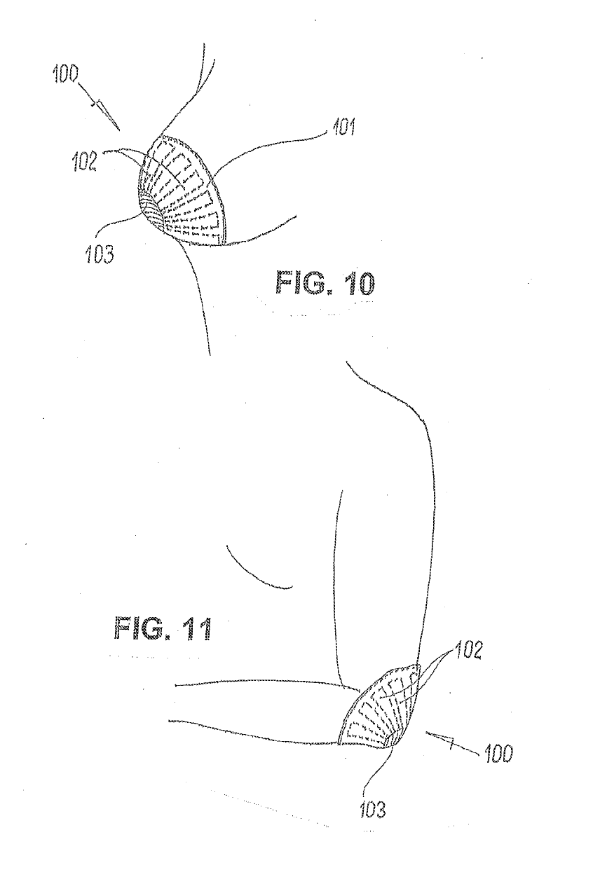

FIGS. 8, 9, 10 and 11 show the placement of a wound care article 100 according to the invention in a bodily region with convex topology, namely, the knee (FIG. 8), the heel (FIG. 9), the breast (FIG. 10) and the elbow (FIG. 11). In this configuration, the wound care article according to the invention can be provided with an adhesive coating on the side facing the wound (in the manner of a border dressing). Alternatively, it can be fixed in place in the traditional manner, for example, with adhesive tape or the like.

This ensures a firm purchase of the wound care article of the invention on the mentioned part of the body, and thus the wound care article is able to make contact with the wound being treated over its entire area.

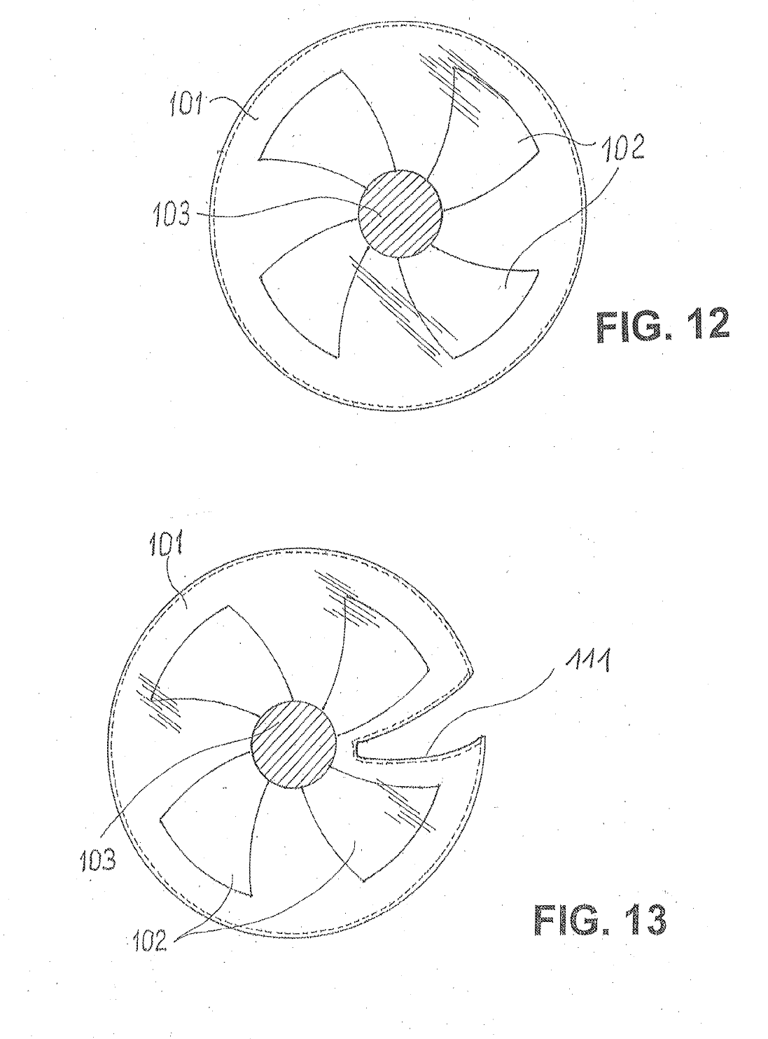

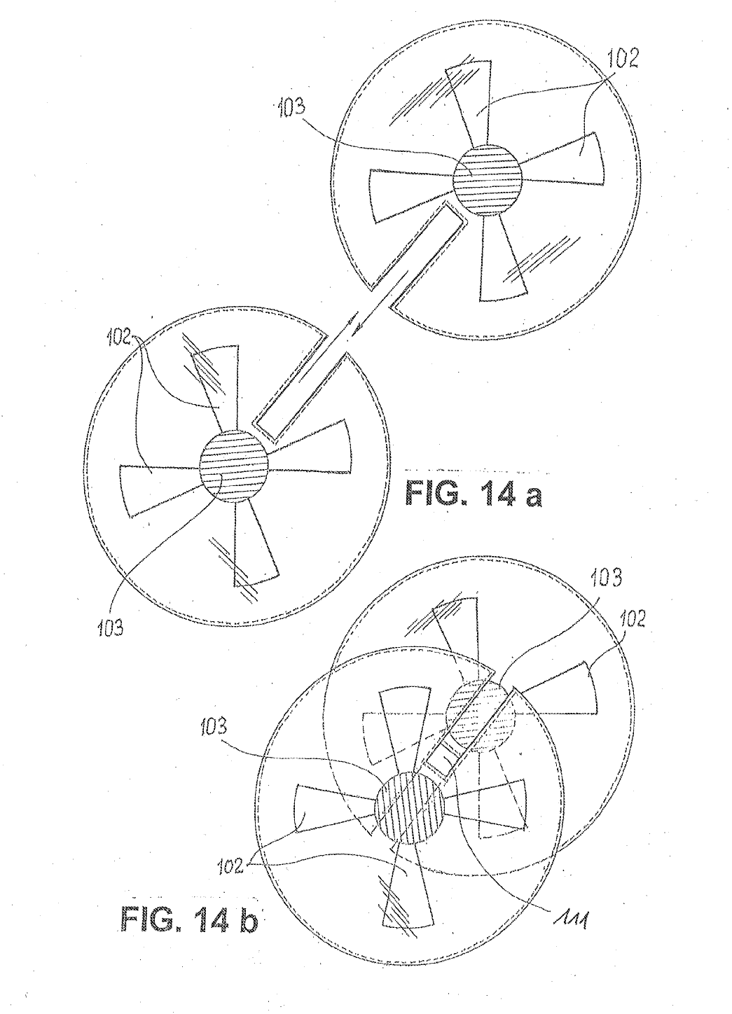

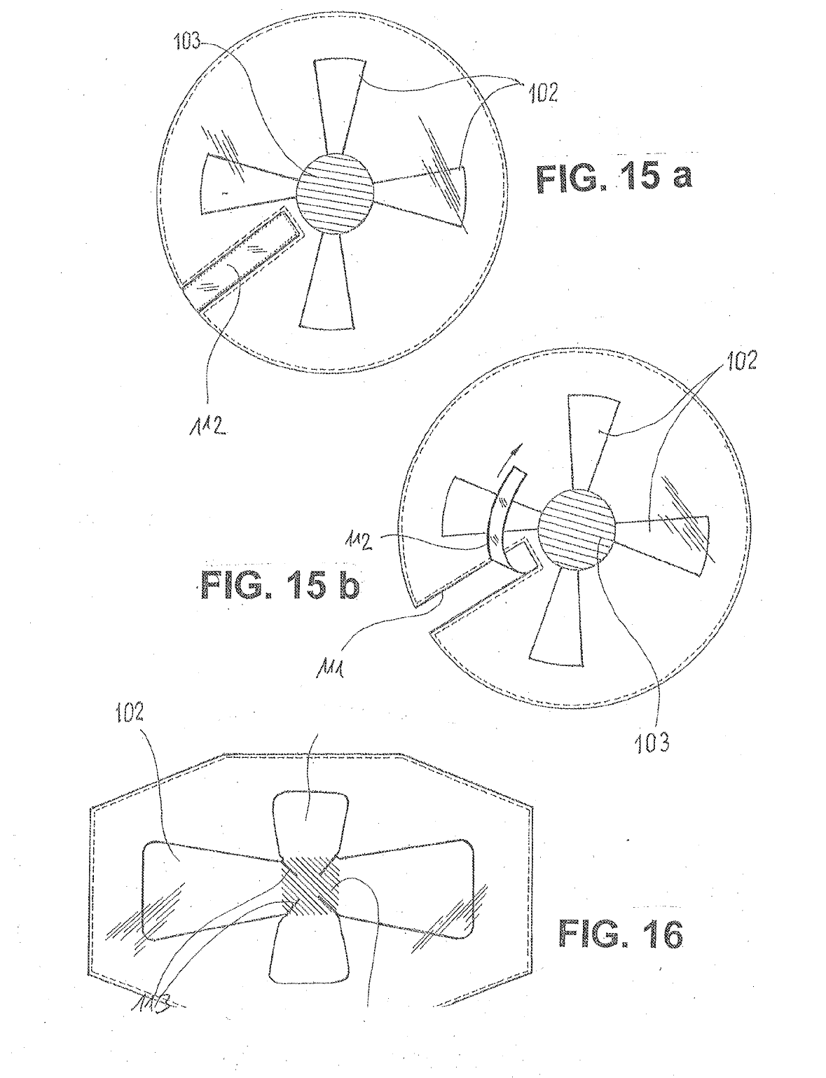

FIG. 12-21 show other possible embodiments, partly with a slot worked into the sheath or a recess (reference number 111) to place the wound care article around a catheter, a drainage tube or a cannula (FIG. 13) or for coupling together two or more wound care articles (FIG. 14a, FIG. 14b) by an overlapping. In departure from FIGS. 13 and 14, the sheath of the wound care article especially for the last mentioned purpose can also have several slots or recesses, as shown in FIG. 19, for example. Said slot or said recess can also be covered by a tear-off section (reference number 112 in FIG. 15 a and b), which is secured by means of perforations to the material of the sheath, so that it is only exposed by tearing off the section 112 when in use.

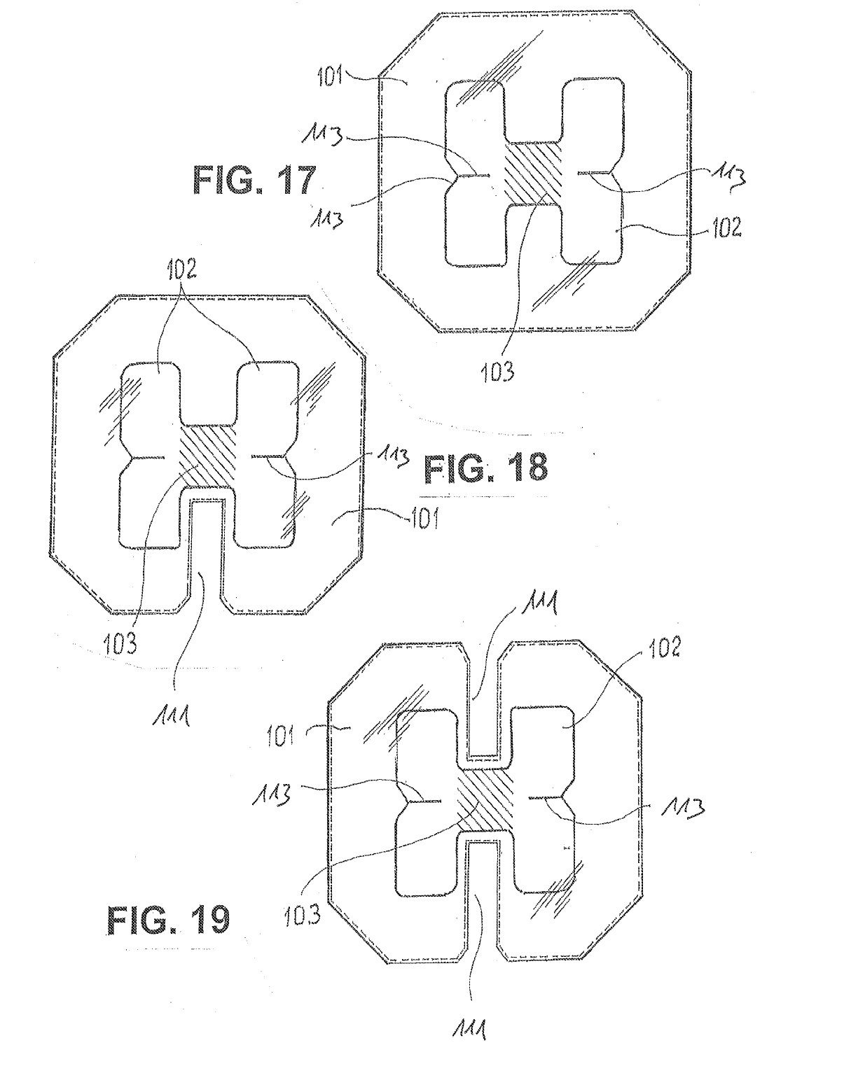

FIG. 17-19 show that the sheet-like layer can also have incisions 113 in its central region. These facilitate the folding of the wound care article, for example, when arranging it in a ditch-like cavity. A similar situation is shown in FIG. 16, where one further notes that the wings of the sheet-like layer can be of different size.

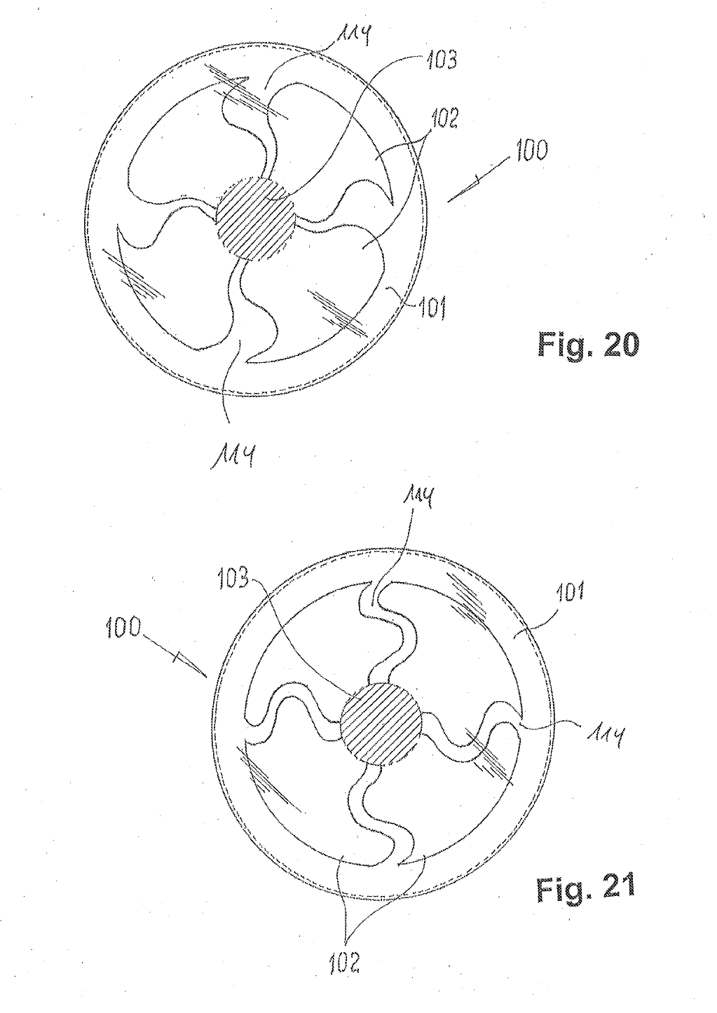

The radial incisions need not have a linear trend. As shown in FIGS. 20 and 21, the radial incisions 114 can also be meandering or corrugated, either with parallel edges or with converging or diverging edges. In this way, when placed in a cavity or a bodily region with convex topology, it is ensured that fluid draining in the region of the incisions sooner or later touches the edges of the wings and is securely taken up. The incisions can also have a crescent shape, as shown in FIGS. 12 and 13.

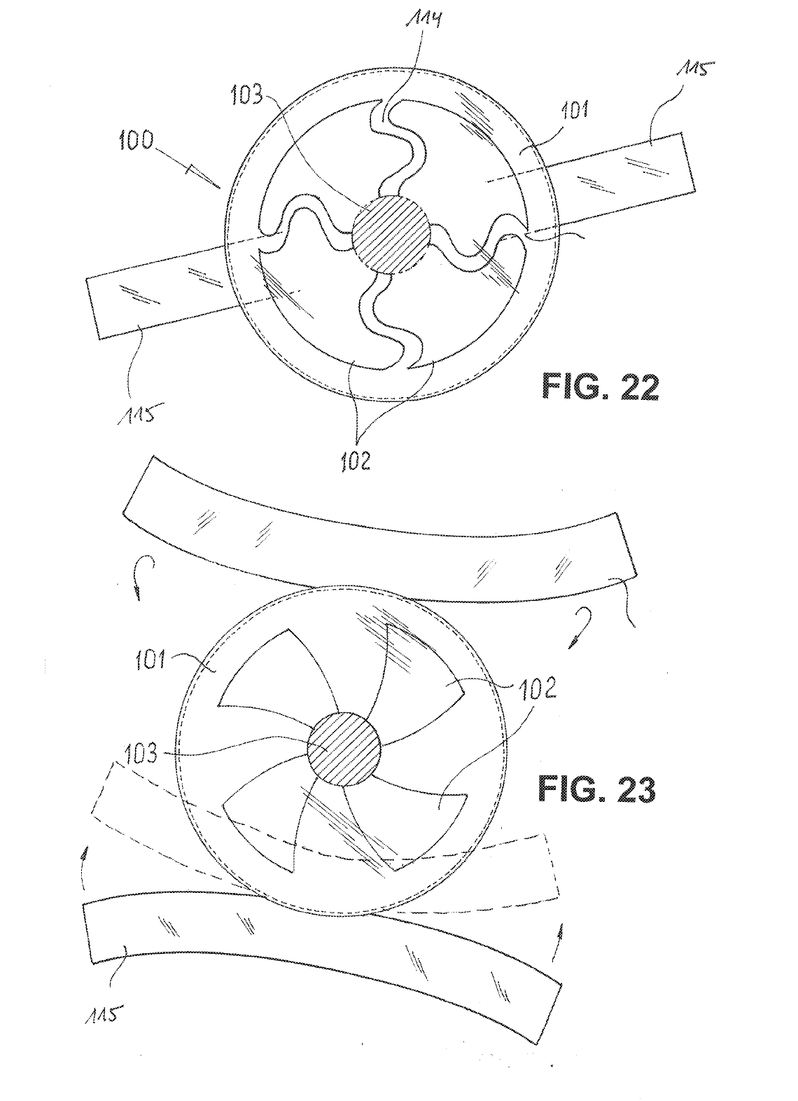

FIGS. 22 and 23 show kits containing at least one wound care article 100 according to one of the preceding claims and at least one glue device. These glue devices can be configured as adhesive strips in the manner of a sticking plaster, being provided in the same or a different packaging than the wound care article. The adhesive strips can be elastic in configuration, have a pull-off protective film and an adhesive layer, such as one of silicone, acrylate, or hydrocolloidal adhesive. By means of the adhesive strips, which can also be fastened to the wound care article and/or form an integral part thereof, the wound care article can be attached to the skin of the patient. For this, the adhesive strips can be arranged in situ central (FIG. 22) or tangential or peripheral (FIG. 23) to the wound care article.

Preferably, the adhesive strips have a curved shape (such as a segment of a circular arc), as shown in FIG. 23. This has special benefits when placing the wound care article in a bodily region with convex topology.

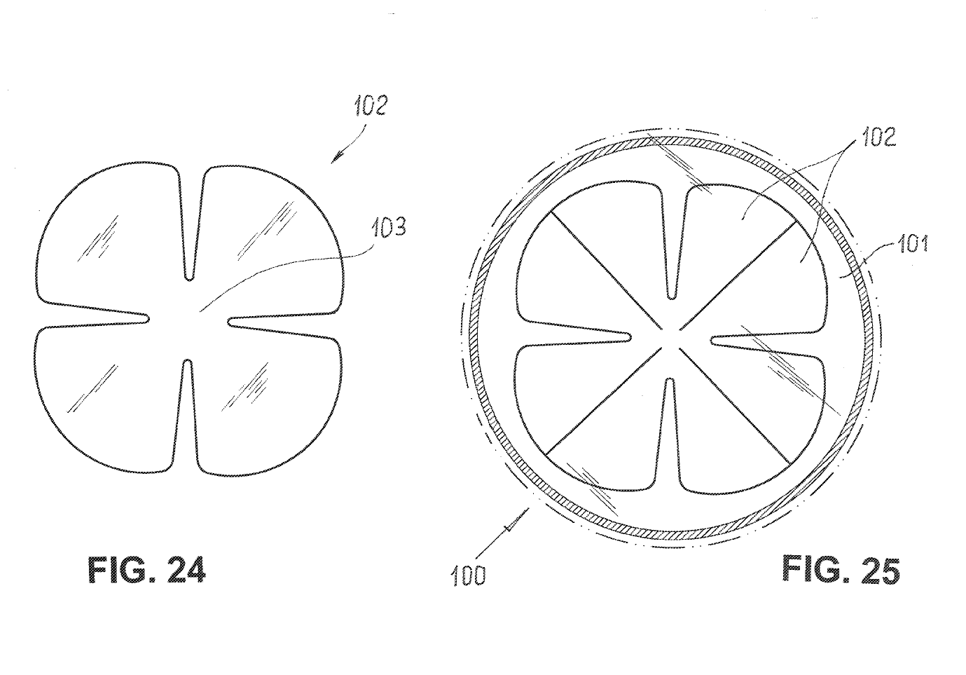

FIGS. 24 and 25 show wound care articles (100) according to the invention with radially symmetrical peripheral shape in the form of a square with rounded corners. This results in a kind of cloverleaf shape. In FIG. 24 the wound care article is shown without and in FIG. 25 with a sheath. In FIG. 25, the wound care article furthermore has incisions as far as the central region, although these can also be omitted.

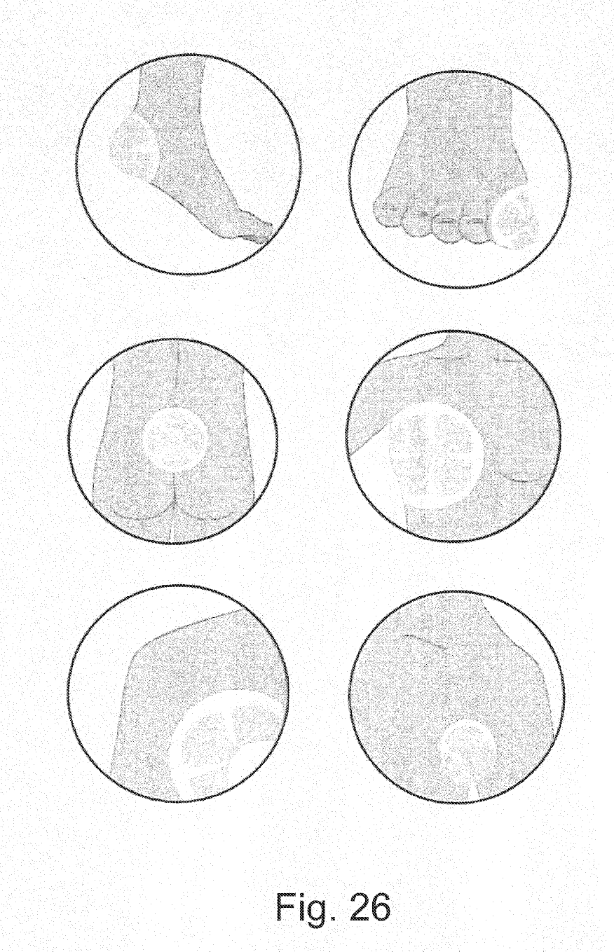

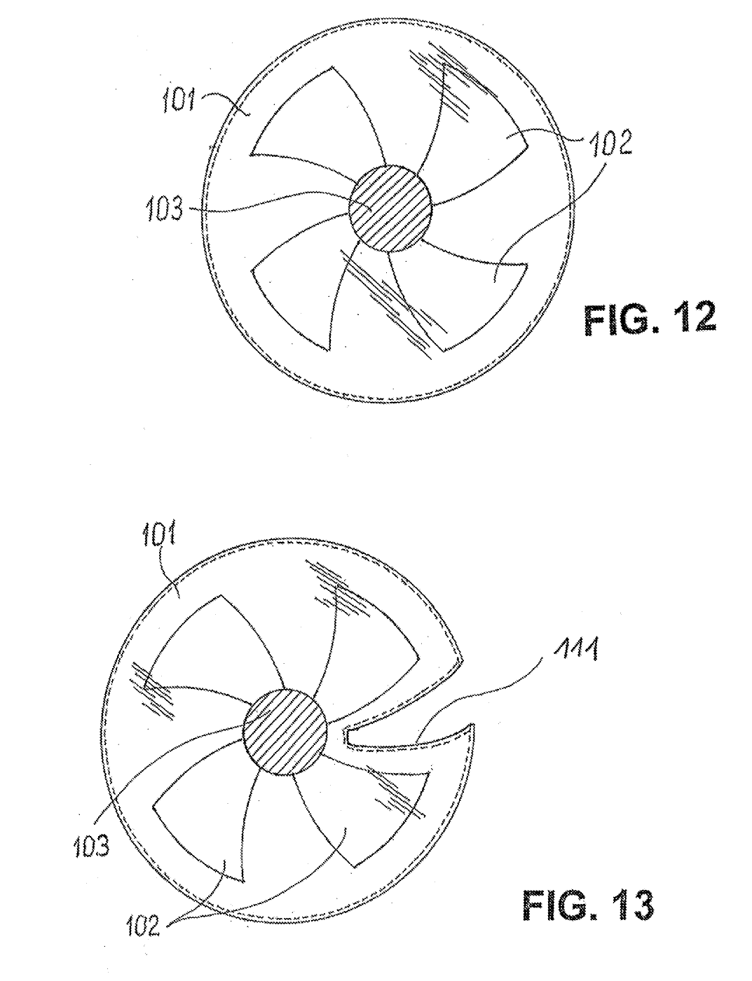

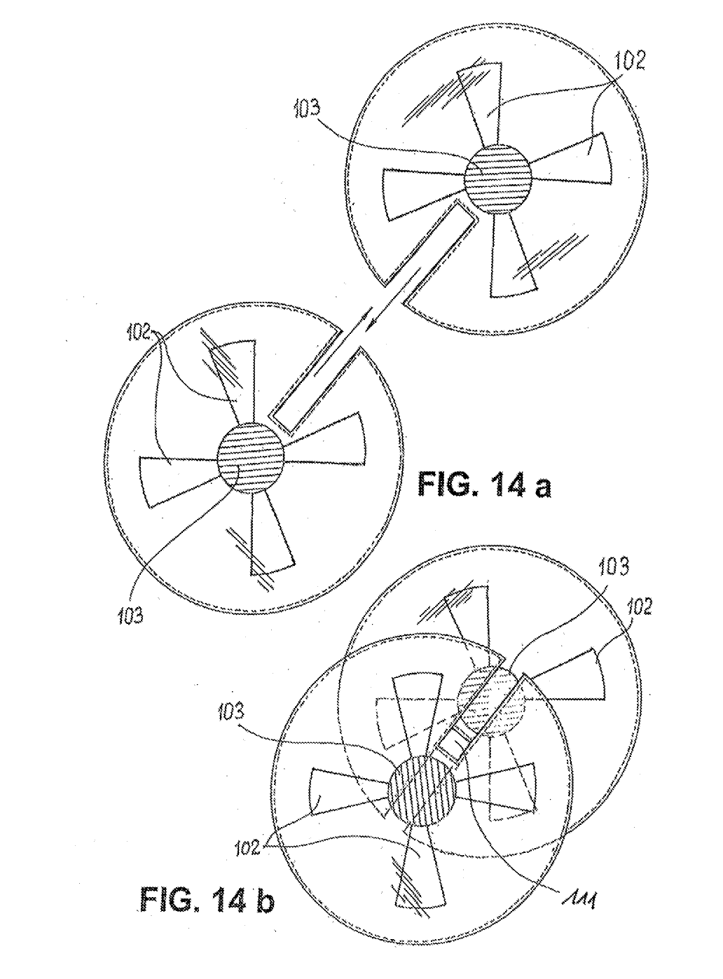

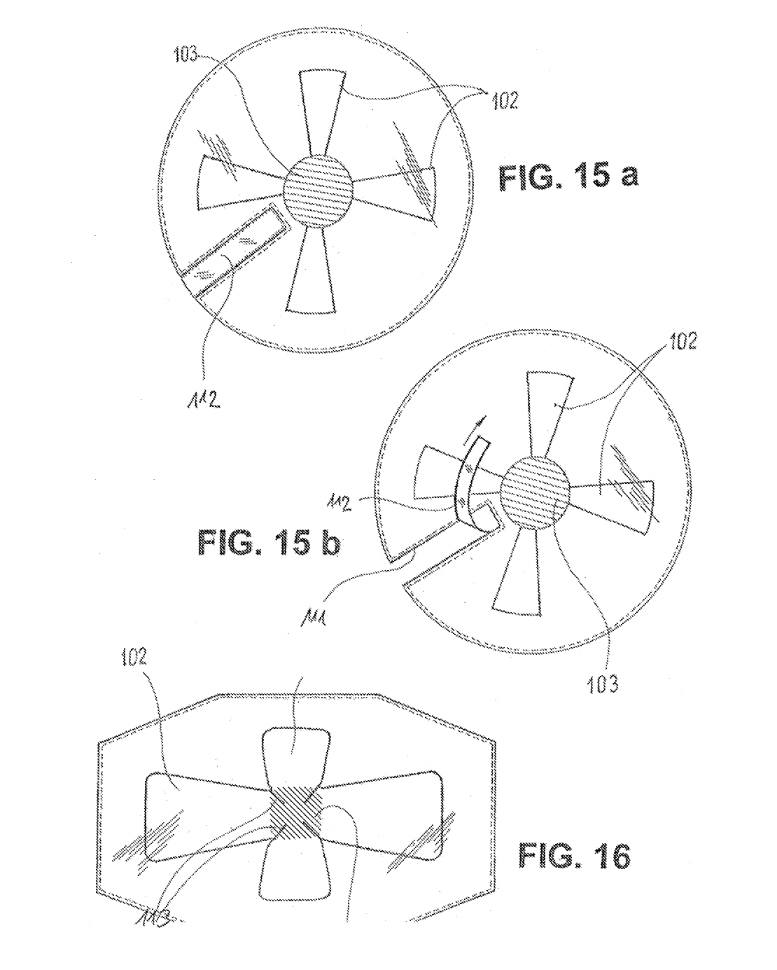

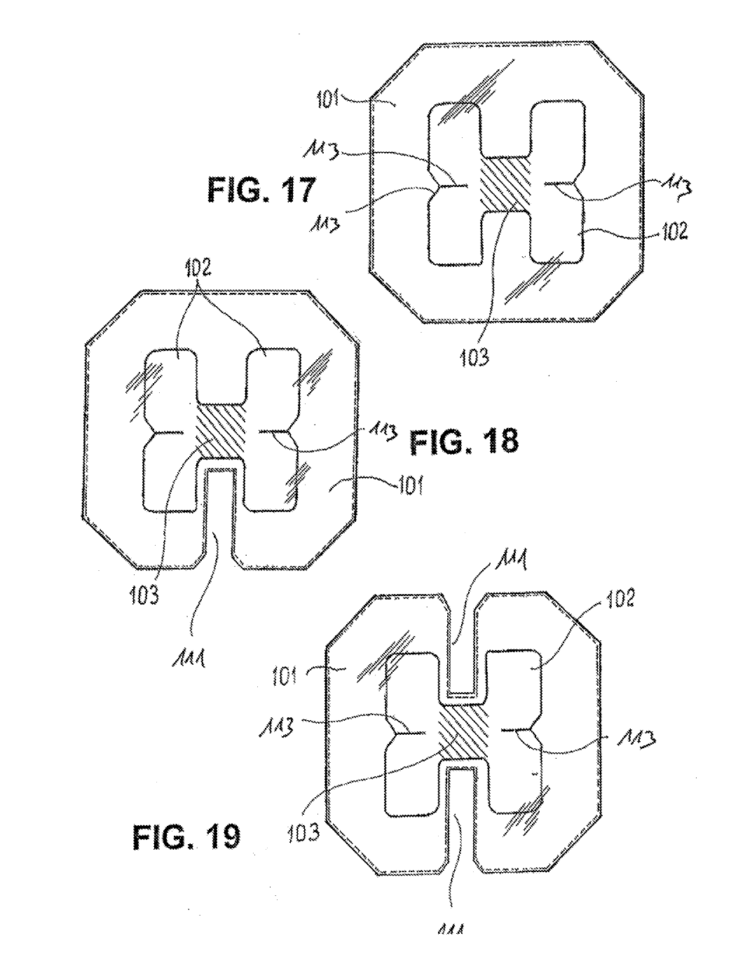

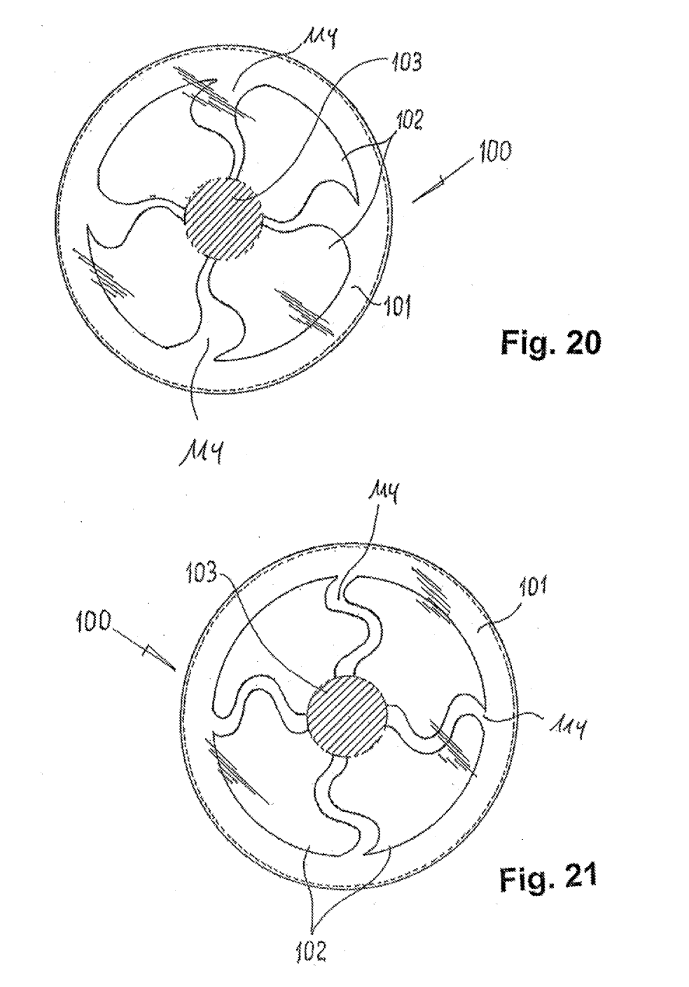

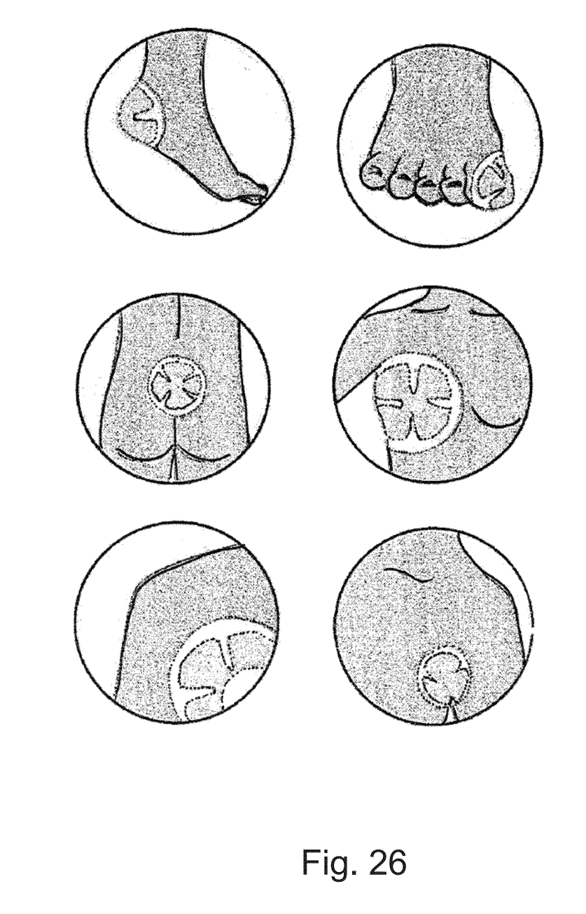

FIG. 26 shows further possible applications of the wound care article of the invention.

### LIST OF REFERENCE NUMBERS

| | |
|---|---|
| Wound care article | 100 |
| Sheath 1 | 101 |
| Wing | 102 |
| Central region | 103 |
| Radial incision | 104 |
| Sheet-like layer | 105 |
| Cut-out region | 106 |
| Lateral incision in the wing | 107 |
| Radial incision in the central region | 108 |
| Central opening | 109 |
| Cavity | 110 |
| Slot | 111 |
| Tear-off section | 112 |
| Incisions in the central region | 113 |
| Meandering/corrugating incisions | 114 |
| Glue device | 115 |

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A wound care article, comprising at least one sheet-like layer, characterized in that the sheet-like layer has at least two wings and a central region, the wings being arranged around the central region and the wings being joined to each other via the central region.

2. The wound care article according to claim 1 for treatment of deep wounds, sores or wounds in body cavities or wounds in regions with convex or irregular topology.

3. The wound care article according to claim 2, characterized in that the sheet-like layer has a radially symmetrical peripheral shape, while the wings are obtained by radial incisions which extend from the margin of the absorbent material up to the central region.

4. The wound care article according to claim 3, characterized in that regions are cut out between the individual wings, so that wings and cut-out regions are arranged uniformly alternating about the central region.

5. The wound care article according to claim 4, characterized in that at least one wing has one or more lateral incisions in the wing.

6. The wound care article according to claim 5, characterized in that the wound care article comprises absorbent material.

7. The wound care article according to claim 6, characterized in that the absorbing material has a nonwoven, preferably an airlaid, and/or the absorbing material has superabsorbing polymers.

8. The wound care article according to claim 7, characterized in that the wound care article has at least in some sections a sheath which is permeable to fluid at least in some sections.

9. The wound care article according to claim 8, characterized in that the wound care article has at least one slot in its sheath.

10. The wound care system according to claim 9, further comprising a drainage device and/or a surgical conduit coupled to the wound care article.

11. A method for treatment of deep wounds and/or wounds of bodily regions with convex topology or wounds situated in the region of an irregular bodily topology comprising:
using a wound care article, comprising at least one sheet-like layer, characterized in that the sheet-like layer has at least two wings and a central region, the wings being arranged around the central region and the wings being joined to each other via the central region.

12. The method of claim 11, further comprising using the wound care article in a partial vacuum wound care system.

13. A kit comprising a wound care article, comprising at least one sheet-like layer, characterized in that the sheet-like layer has at least two wings and a central region, the wings being arranged around the central region and the wings being joined to each other via the central region, and operably coupled to at least one glue device.
